(19) United States
(12) Patent Application Publication
(10) Pub. No.: US 2014/0058227 A1
(43) Pub. Date: Feb. 27, 2014

(54) MEASURING DEVICE, AND MEASURING METHOD

(71) Applicants: Mikihiro Yamanaka, Osaka-shi (JP); Megumi Hijikuro, Osaka-shi (JP); Keita Hara, Osaka-shi (JP)

(72) Inventors: Mikihiro Yamanaka, Osaka-shi (JP); Megumi Hijikuro, Osaka-shi (JP); Keita Hara, Osaka-shi (JP)

(73) Assignee: SHARP KABUSHIKI KAISHA, Osaka-shi, Osaka (JP)

(21) Appl. No.: 14/116,369

(22) PCT Filed: Apr. 10, 2012
(86) PCT No.: PCT/JP2012/059803

§ 371 Date: Nov. 8, 2013

(30) Foreign Application Priority Data
May 26, 2011 (JP) 2011-118346

(51) Int. Cl. A61B 5/00; A61B 5/145; A61B 5/1455

(52) U.S. Cl. CPC A61B 5/6834; A61B 5/1455; A61B 5/14532
USPC 600/316; 600/317

(57) ABSTRACT

Provided are a transmissive suction mechanism (1), a suction hole (5), provided in the suction mechanism (1), through which skin is pulled into the suction mechanism (1), and an exhaust hole (4), provided in the suction mechanism (1), through which air in the suction mechanism (1) is removed so as to reduce the pressure in the suction mechanism (1).

### TECHNICAL FIELD

The present invention relates to a skin sampling member that samples skin, a measuring device including the skin sampling member, and a measuring method using the skin sampling member.

### BACKGROUND ART

Hitherto, anti-glycation (anti-aging) cosmetics aimed at reducing advanced glycation endproducts (AGEs) which accumulate in skin have been commercialized. AGEs are end products produced via non-enzymatic glycosylation reaction (Maillard reaction) between protein and carbohydrate or lipid. AGEs are yellowish brown in color, and some of them are fluorescent materials. In addition, AGEs have a property of forming crosslink by being combined with structural protein that is present in the vicinity thereof. In particular, crosslink between AGEs and collagen constituting dermis problematically reduces elasticity of the skin and also causes dullness of the skin.

As methods of evaluating a state of the skin, a method of measuring an amount of moisture or an amount of oil of a sebum layer or a horny layer of the skin, and a method of measuring a surface electric potential are known. However, there is a problem in that both the methods are nothing more than evaluating information of a skin surface.

As another method of evaluating a state of the skin, there is a skin diagnosis method which is disclosed in Patent Literature (PTL) 1.

In this diagnosis method, a horny cell layer, which is extracted from the skin through a tape stripping method of sampling corneocytes by using an adhesive tape, is irradiated with ultraviolet rays, an abundance ratio of β sheet-type keratin in the horny cell layer is estimated, and/or skin flexibility is diagnosed in accordance with the intensity of fluorescence caused by irradiation of the ultraviolet rays.

Meanwhile, examples of other methods of evaluating a state of the skin include techniques disclosed in PTL 2 and PTL 3. In the techniques disclosed in PTL 2 and PTL 3, a skin sample is irradiated with light and light reflected from the skin sample is detected.

### CITATION LIST

### Patent Literature

PTL 1: Japanese Unexamined Patent Application Publication No. 2005-348991 (published on Dec. 22, 2005)
PTL 2: Japanese Unexamined Patent Application Publication No. 2004-290234 (published on Oct. 21, 2004)
PTL 3: Pamphlet of International Publication WO 01/22869 A1 (published on Apr. 5, 2001)

### SUMMARY OF INVENTION

### Technical Problem

However, the diagnosis method disclosed in PTL 1 mentioned above has a problem that acquired knowledge of skin may be applied to corneocytes alone. In addition, the diagnosis method also has a problem that a procedure from the extraction of the corneocytes to the measurement of fluorescence from the corneocytes is complicated.

For example, the diagnosis method disclosed in PTL 1 mentioned above includes at least four processes:
(1) a process of extracting corneocytes from the skin by using an adhesive tape,

(2) a process of melting the adhesive tape by using an organic solvent over half or more days,

(3) a process of preparing a sample by using the obtained corneocytes, and

(4) a process of measuring fluorescence of the prepared sample by using a fluorescence spectrophotometer.

For this reason, there is a problem that a procedure for acquiring knowledge of a state of skin is very complicated for an examinee.

In addition, in the above-mentioned diagnosis method, a diagnosis result of the skin is not obtained if one or more days have not elapsed after the examinee feels a desire to confirm his or her skin state. Therefore, when cosmetic counseling is performed, the counseling has to be performed one or more days after skin tissues have been extracted. For this reason, a timely consultation based on a diagnosis result of his or her skin cannot be conducted and thus proper counseling cannot be available.

On the other hand, since the techniques disclosed in PTL 2 and PTL 3 have a simple configuration in which only reflected light from skin has to be measured, the procedure thereof is simple. However, both the techniques have a problem that it is difficult to evaluate a state of the skin including a deeper portion in an epidermal layer or a dermic layer in the skin.

The present invention is contrived in view of such situations, and an object thereof is to provide a skin sampling member capable of simply sampling a portion of skin, a measuring device including the skin sampling member, and a measuring method using the skin sampling member, for the purpose of confirming a state of the skin including at least an epidermal layer or a dermic layer.

### Solution to Problem

To solve the above-mentioned problem, the skin sampling member of the present invention includes a housing that is made of a transmissive material, a suction hole, provided in the housing, through which skin is pulled into the housing, and an exhaust hole, provided in the housing, through which air in the housing is removed so as to reduce the pressure in the housing.

According to the above-mentioned configuration, it is possible to pull a specific portion (measurement object) of a living body into the housing through the suction hole by removing air in the housing through the exhaust hole so as to reduce the pressure in the housing after bringing the suction hole into contact with the measurement object. Therefore, it is possible to sample the specific portion of the skin through a simple procedure.

Examples of the measurement object can include an arm, a wrist, an earlobe, a fingertip, a palm, a cheek, the inner side of an upper arm of an examinee, and the like.

In addition, since the housing has transmittance, the portion of the skin (the specific portion of the skin) which is pulled into the housing is irradiated with light, and thus it is possible to optically measure light generated from the portion of the skin being irradiated with light.

Meanwhile, examples of the light generated from the portion of the skin being irradiated with light may include reflected light of the light with which the portion of the skin is irradiated, transmitted light, passing through the skin, with which the portion of the skin is irradiated, or fluorescence generated from the portion of the skin being irradiated with excitation light (light).

In addition, in order to perform the above-mentioned optical measurement, a portion of skin which is sampled has only to be irradiated with light. Thus, it is possible to simplify a procedure of confirming a state of the skin as compared with the diagnosis method disclosed in PTL 1.

Furthermore, in spite of a simple method of pulling a portion of skin into a housing, an epidermal layer or a dermic layer can also be included in the pulled portion of the skin when the internal volume of the housing is properly increased.

As described above, it is possible to simply sample a portion of skin for the purpose of confirming a state of the skin including at least an epidermal layer or a dermic layer.

### Advantageous Effects of Invention

As described above, a skin sampling member of the present invention includes a housing that is made of a transmissive material, a suction hole, provided in the housing, through which skin is pulled into the housing, and an exhaust hole, provided in the housing, through which air in the housing is removed so as to reduce the pressure in the housing.

For this reason, an effect is exhibited in which it is possible to simply sample a portion of skin for the purpose of confirming a state of the skin including at least an epidermal layer or a dermic layer.

Other objects, features, and advantages of the present invention will be sufficiently known by the following description. In addition, the advantages of the present invention will be apparent from the following description with reference to the accompanying drawings.

### BRIEF DESCRIPTION OF DRAWINGS

FIG. 1 is a diagram illustrating the whole configuration of a measuring device according to an embodiment of the present invention.

FIG. 2 is a diagram illustrating a contour of a suction mechanism (skin sampling member) according to an embodiment of the present invention; FIG. 2(a) is a perspective view illustrating a contour of the suction mechanism (skin sampling member) and FIG. 2(b) is a side view illustrating a contour of the suction mechanism.

FIG. 3 is a diagram schematically illustrating the suction mechanism having an internal volume being variable.

FIG. 4 is a diagram illustrating an example (portable type) of the measuring device.

FIG. 5 is a diagram illustrating another example (ear sensor type) of the measuring device.

FIG. 6 is a diagram illustrating a relationship between a wavelength and intensity (detection intensity) of fluorescence in a specific portion of a living body.

FIG. 7 is a diagram illustrating the absorbance of hemoglobin at each wavelength.

FIG. 8 is a diagram illustrating a state when a portion of a surface of the suction mechanism on the fluorescence measurement side is shielded from light.

FIG. 9 is a diagram illustrating a state when a portion of a surface of the suction mechanism on the fluorescence measurement side is shielded from light.

FIG. 10 is a diagram illustrating a cross-section of skin, and thicknesses and turnovers of a horny layer, an epidermis (layer), and a dermis (layer); FIG. 10(a) illustrates the cross-section of the skin and FIG. 10(b) illustrates the thicknesses and turnovers of the horny layer, the epidermis (layer), and the dermis (layer).

### DESCRIPTION OF EMBODIMENTS

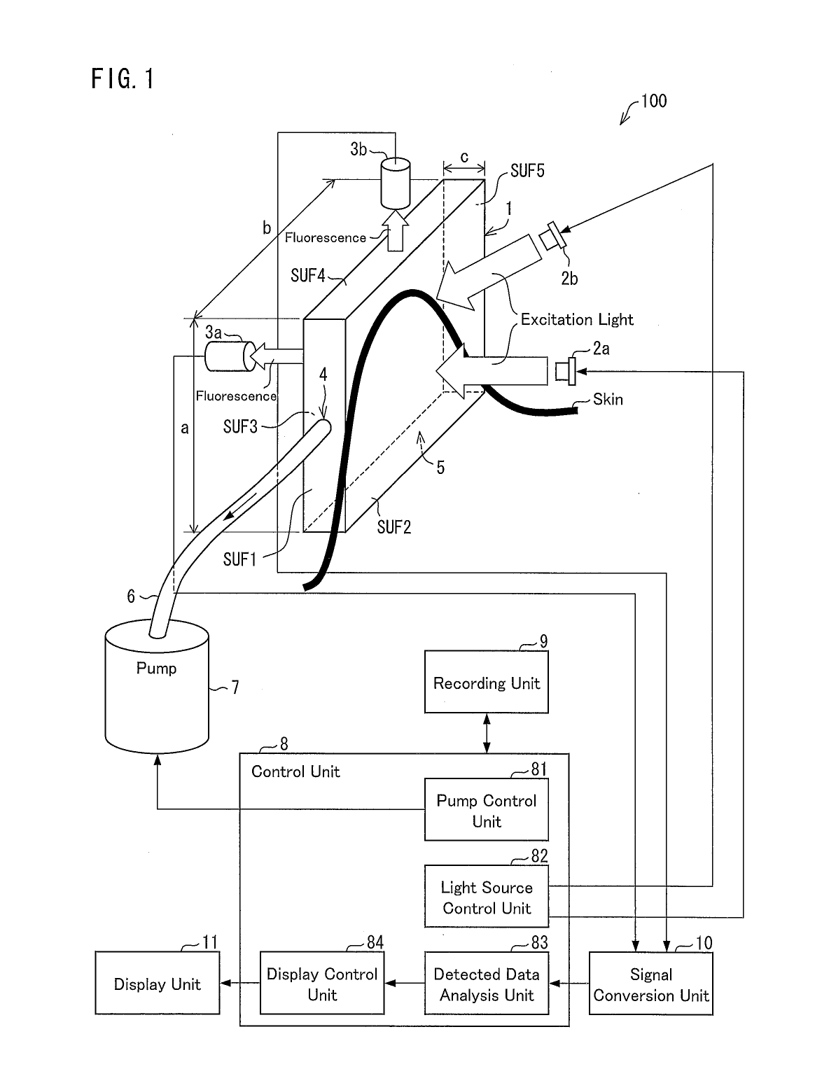

An embodiment of the present invention will be described below with reference to FIGS. 1 through 10. Description of configurations other than a configuration described in a certain paragraph below is sometimes omitted if necessary. However, those described in other paragraphs are the same as the configurations. In addition, for convenience of description, members having the same function as those described in each paragraph are denoted by the same reference numerals, and the description thereof will not be repeated appropriately.

1. Measuring Device

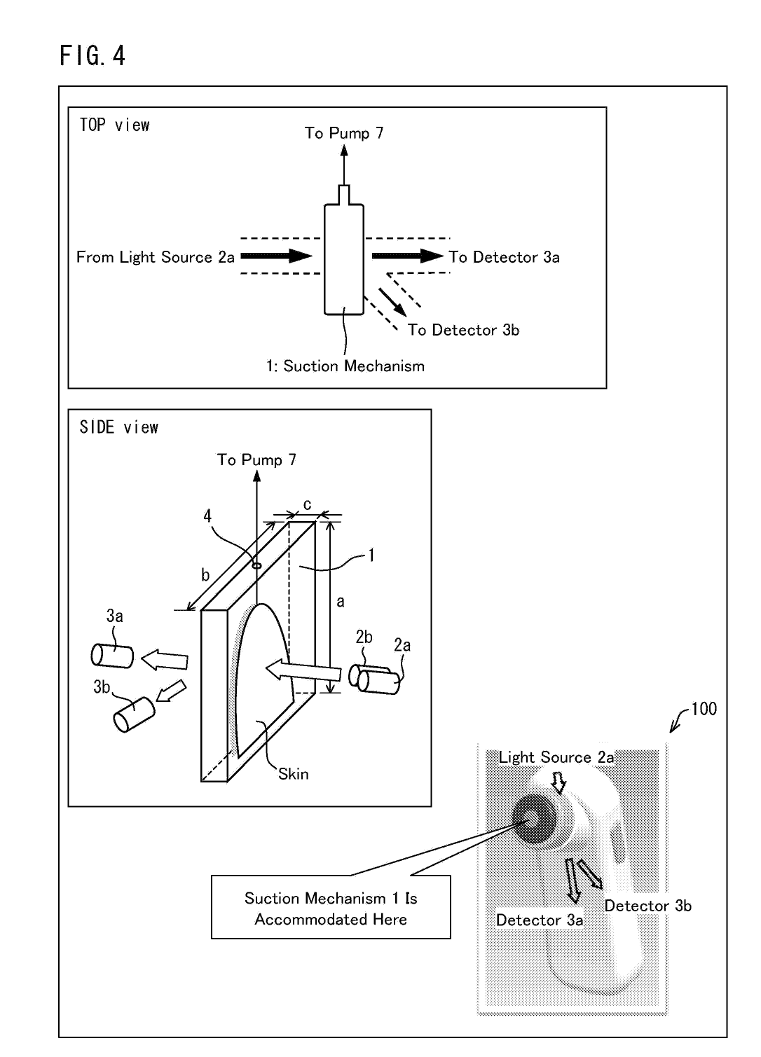

First, the whole configuration of a measuring device 100 according to an embodiment of the present invention will be described with reference to FIGS. 1 and 4. FIG. 1 is a diagram illustrating the whole configuration of the measuring device 100. In addition, FIG. 4 is a diagram illustrating an example (hereinafter, referred to as a portable type measuring device) of the measuring device 100.

Meanwhile, in this embodiment, the measuring device 100 will be described which detects (specifies) the intensity of fluorescence that is obtained from a portion (or a measurement object) of skin of an examinee as an object to be measured.

However, data specified by the measuring device 100 is not limited to the intensity of fluorescence, and the measuring device may be configured to specify other pieces of physical property information (or physical quantities).

For example, in general, examples of light generated by a portion of skin being irradiated with light can include reflected light of the light with which the portion of the skin is irradiated, transmitted light, passing through the skin, with which the portion of the skin is irradiated, or fluorescence (fluorescence derived from a material contained in the skin) which is generated by the irradiation of excitation light (light).

Therefore, the measuring device 100 may specify not only the intensity of light as described in this embodiment, but also any one of pieces of physical property information (or physical quantities) such as a half-value width thereof, a wavelength of detected light, the reflectivity of the skin, or the transmittance of the skin, which are derived from a material contained in the portion of the skin.

As illustrated in FIG. 1, the measuring device 100 includes a suction mechanism (skin sampling member, housing) 1, a light source 2a, a light source (another light source) 2b, detectors (light detection units) 3a and 3b, a duct 6, a pump 7, a control unit 8, a recording unit 9, a signal conversion unit 10, and a display unit 11.

Next, a contour of a measurement of a portable type measuring device is illustrated on the lower right side of FIG. 4. Meanwhile, in FIG. 4, an example of the arrangement of the suction mechanism 1, the light source 2a, and the detectors 3a and 3b in the portable type measuring device is illustrated.

In addition, “TOP view” of FIG. 4 illustrates an example of the arrangement of the suction mechanism 1, the light source 2a, and the detectors 3a and 3b when the portable type measuring device is viewed from above. Meanwhile, the suction mechanism 1 illustrated in FIG. 4 is different from the suction mechanism 1 illustrated in FIG. 1 in terms of the position of a suction hole 5. The suction hole 5 is provided in a position (the upper side of FIG. 4) which is opposite to an exhaust hole 4 (the lower side of FIG. 4). In this manner, the suction hole 5 of the suction mechanism 1 may be provided in a position opposite to the exhaust hole 4.

On the other hand, “SIDE view” of FIG. 4 illustrates an example of the arrangement of the suction mechanism 1, the light source 2a, the detectors 3a and 3b, and the pump 7 when the inside of the portable type measuring device is viewed from an oblique lateral direction.

Suction Mechanism 1

As illustrated in FIG. 1, the suction mechanism 1 of this embodiment has a substantially rectangular parallelepiped shape. However, the shape of the suction mechanism 1 is not limited to the substantially rectangular parallelepiped shape. The suction mechanism may have any shape as long as it is a shape capable of pulling a portion of skin. For example, the suction mechanism may employ various shapes such as a substantially rectangular parallelepiped shape, a substantially cube shape, a truncated pyramid shape, a circular truncated cone shape, or such a shape that corners of each of those shapes are rounded.

In addition, among six surfaces of the suction mechanism 1, five surfaces are referred to as a surface SUF1 to a surface SUF5, respectively. In addition, the remaining one surface side (the lower side of FIG. 1) of the substantially rectangular parallelepiped shape is opened, and forms the suction hole 5 for pulling skin into therein. Furthermore, the surface SUF1 is provided with the exhaust hole 4 so as to be connected with the pump 7 through the duct 6. That is, the exhaust hole 4 is a hole for exhausting (decompressing the inside of the suction mechanism 1) gas (for example, air) from the inside of the suction mechanism 1.

A material for forming the suction mechanism 1 of this embodiment is a transmissive quartz glass. For example, the suction mechanism is manufactured by opening a hole (the exhaust hole 4) in a portion of a quartz cell container. Meanwhile, the material for forming the suction mechanism 1 is not limited to quartz glass, and may be a transmissive resin material or ceramic.

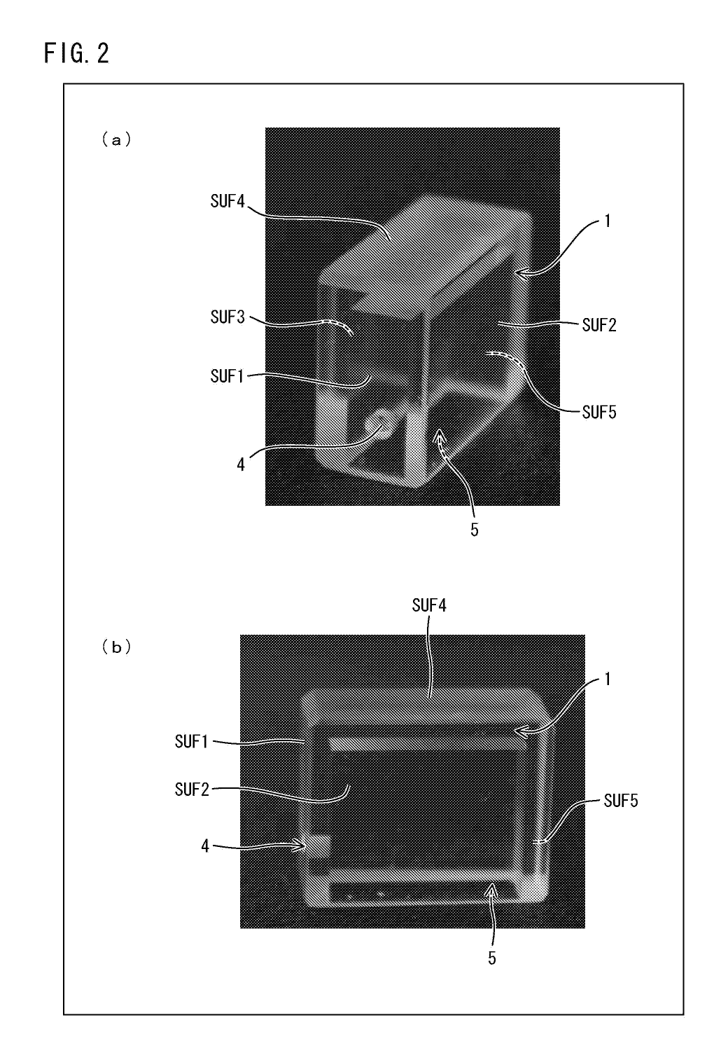

An example (quartz cell container) of the suction mechanism 1 is illustrated in FIG. 2. FIG. 2(a) is a perspective view illustrating a contour of the suction mechanism 1. FIG. 2(b) is a side view illustrating a contour of the suction mechanism 1.

As illustrated in FIG. 1, the suction mechanism 1 of this embodiment has a length a of approximately 20 mm, a length b of approximately 25 mm, and a length c of approximately 10 mm (for example, a thickness of a quartz plate constituting the quartz cell container illustrated in FIG. 2 is neglected).

When an internal volume of the suction mechanism 1 is constant, a negative pressure of the inside of the suction mechanism 1 may be increased in order to increase an amount of skin to be suctioned.

However, when the negative pressure of the inside of the suction mechanism 1 is increased, the length a is required to be equal to or greater than 2 mm to 5 mm and both the length b and the length c are required to be equal to greater than at least 4 mm to 10 mm in order for a portion of the skin which is suctioned into the suction mechanism 1 to include at least a dermic layer. For example, it is considered that a half an amount of skin to be sampled by the suction hole 5 having the length b and the length c corresponds to a thickness (depth) of a cross-section of the skin to be suctioned.

At this time, since the dermic layer has a thickness of approximately 2 mm to 5 mm, the length a is required to be at least approximately 2 mm to 5 mm and the length b and the length c are required to be approximately twice the thickness of the dermic layer, that is, at least approximately 4 mm to 10 mm.

From the same point of view, when the negative pressure of the inside of the suction mechanism 1 is increased, the internal volume of the suction mechanism 1 is required to be equal to or greater than at least 32 mm³ to 500 mm³ in order for the portion of the skin which is suctioned into the suction mechanism 1 to include at least a dermic layer.

When the internal volume of the suction mechanism 1 is constant, a magnitude of internal pressure of the suction mechanism 1 may be adjusted in order to adjust an amount of skin to be sampled. At this time, although there is an upper limit according to the internal volume of the suction mechanism 1, the higher the pressure is, the greater the amount of skin to be sampled is, and the lower the pressure is, the smaller the amount of skin to be sampled is.

On the other hand, when the internal pressure of the suction mechanism 1 is constant, as a method of adjusting the amount of skin to be sampled, the following methods are considered,

(1) a method of preparing a plurality of the suction mechanisms 1 having different internal volumes so as to be exchangeable with each other,

(2) a method of making the internal volume of the suction mechanism 1 itself variable, and the like.

In the case of (1) mentioned above, examples of a method of changing the internal volume of the suction mechanism 1 include a method of changing the size of the whole suction mechanism 1, a method of, when the suction mechanism 1 is constituted by a quartz cell container, increasing or decreasing the thickness of the quartz plate constituting the quartz cell container, and the like.

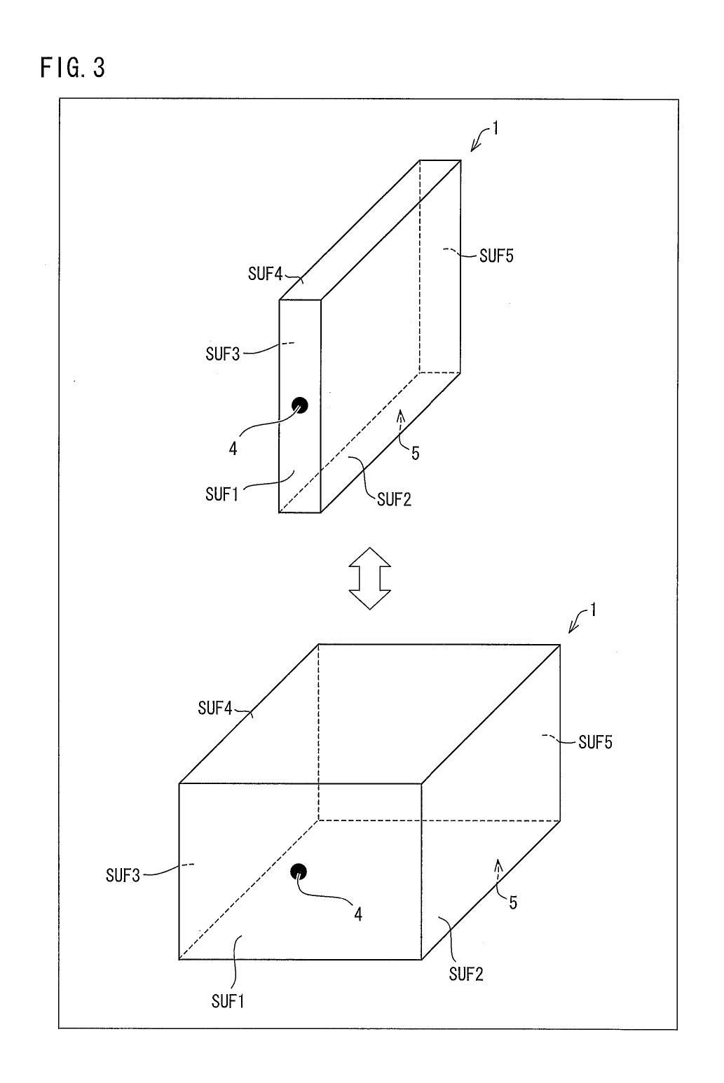

On the other hand, in the case of (2) mentioned above, a method is considered of providing a structure of the suction mechanism 1 in which the internal volume is variable. For example, FIG. 3 schematically illustrates an example of the suction mechanism 1 having a variable internal volume.

As described above, it is possible to change an amount of skin to be sampled by causing the suction mechanism 1 to have a variable internal volume. Thus, it is possible to select whether to sample any of the surface of the skin to a region of a horny layer, a region of an epidermal layer, and a region of a dermic layer at approximately the same location.

Incidentally, in the diagnosis method disclosed in PTL 1 mentioned above, although it is possible to confirm a state of the corneocytes (horny layer), there is an additional problem that it is not possible to confirm states or a state of the epidermal layer and/or the dermic layer.

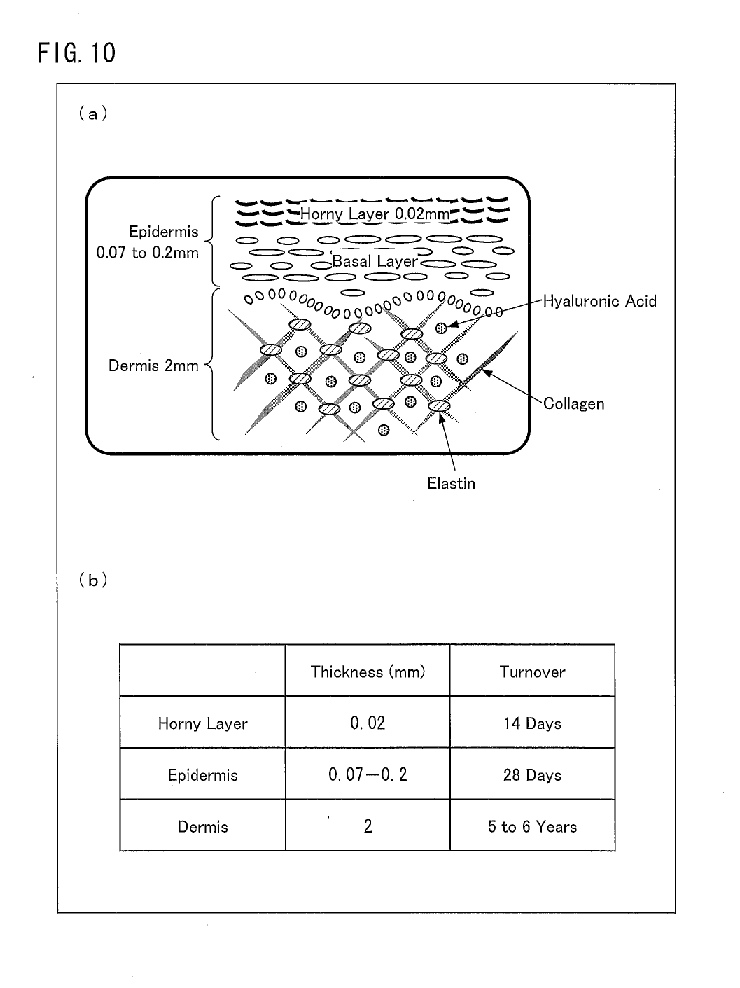

For example, elasticity of the skin is influenced not only by a state of the horny layer having a thickness of only 0.02 mm illustrated in FIG. 10(a) but also by a state of the epidermal layer (thickness of 0.07 mm to 0.2 mm), and furthermore, a state of the dermic layer (thickness of 2 mm). Equal to or greater than 70% of the dermic layer is formed of collagen fiber, but advanced glycation endproducts (AGEs) are cross-linked with the collagen fiber. A three-dimensional network structure of the collagen fiber collapses, and fibroblasts, hyaluronic acid, and the like are reduced, due to the crosslink through the AGEs. In addition, a structure of a basal layer at a boundary between the epidermal layer and the dermic layer collapses due to the crosslink through the AGEs, and thus the boundary between the dermic layer and the epidermal layer becomes unclear. As a result, the elasticity of the skin is decreased, and dullness of the skin progresses from the problem of a color tone of the AGEs.

In addition, as illustrated in FIG. 10(b), the horny layer repeats a turnover with a period of 14 days, while the epidermis and the dermis repeat a turnover with a period of 28 days and a period of 5 to 6 years, respectively. Therefore, it is obvious that health status of the epidermis and the dermis cannot be neglected in skin care.

Consequently, in order to solve the above-mentioned additional problem, the internal pressure (or volume) of the suction mechanism 1 may be at least variable from a first magnitude to a second magnitude described below. Alternatively, the internal pressure (or volume) of the suction mechanism 1 may be at least variable from the first magnitude to a third magnitude.

Here, the first magnitude is such a magnitude that most of a portion of skin is constituted by a horny layer. In addition, the second magnitude is such a magnitude that most of a portion of skin is constituted by a horny layer and an epidermal layer. Furthermore, the third magnitude is such a magnitude that a portion of skin includes at least a dermic layer.

Therefore, when the internal pressure (or volume) of the suction mechanism 1 has the first magnitude, most of a portion of skin which is suctioned into the suction mechanism 1 can be constituted by a horny layer. In addition, when the internal pressure (or volume) of the suction mechanism 1 has the second magnitude, most of a portion of skin which is suctioned into the suction mechanism 1 can be constituted by a horny layer and an epidermal layer. Furthermore, when the internal pressure (or volume) of the suction mechanism 1 has the third magnitude, a portion of skin which is suctioned into the suction mechanism 1 can include at least a dermic layer.

Thus, for example, it is possible to measure AGEs-derived fluorescence present in the horny layer, the epidermal layer, and/or the dermic layer. For this reason, the intensity of fluorescence to be detected is associated in advance with an amount of AGEs present in the horny layer, the epidermal layer, and/or the dermic layer, and thus it is also possible to specify the amount of AGEs present in the horny layer, the epidermal layer, and/or the dermic layer. In addition, according to the above-mentioned configuration, it is also possible to know which part is glycosylated in the horny layer, the epidermal layer, and/or the dermic layer, and thus it is possible to use the measuring device at the scene of counseling such as confirmation of a cosmetic effect.

Next, in order for the internal volume of the suction mechanism 1 itself to be variable, the whole suction mechanism 1 may be formed of a transmissive or elastic material (for example, silicone rubber). Alternatively, some of portions of six surfaces of the suction mechanism 1 may be formed of an elastic material (for example, silicone rubber), and the remaining portions may be formed of quartz glass, a rigid resin material, ceramic, or the like.

For example, among six surfaces of the suction mechanism 1, portions of at least a set of a surface (surface on the side irradiated with light) SUF2 and a surface (surface on the opposite side) SUF3, which are opposite to each other, are formed of a transmissive rigid resin material. On the other hand, portions (elastic portions) of a surface SUF1 for coupling the surface SUF2 and the surface SUF3 to each other, a surface SUF4, and a surface SUF5 are formed of silicone rubber or the like.

Thus, a distance between the side (side of the surface SUF2) of the suction mechanism 1 which is at least irradiated with light and the side (side of the surface SUF3) which is opposite thereto becomes variable due to the presence of the elastic portions. In other words, the internal volume of the suction mechanism 1 becomes variable. For this reason, it is possible to adjust an amount of skin to be suctioned into the suction mechanism 1.

Therefore, it is possible to measure fluorescence from the horny layer, the epidermal layer, and/or the dermic layer of the skin on the basis of an amount of skin to be sampled. In addition, according to the above-mentioned configuration, it is also possible to measure the amount of AGEs present in the horny layer, the epidermal layer, and/or the dermic layer of the skin which are present in a direction (depth direction from a surface of the skin) along a cross-section of the skin at approximately the same location.

Products on the market such as “KJR632” manufactured by Shin-Etsu Chemical Co., Ltd. can be used as silicone raw material composition. The silicone raw material composition may have a filler, a heat-resistance material, a plasticizer, or the like added, to the extent that the intensity or transparency of a silicone resin to be obtained is not damaged, in addition to the above-mentioned component.

In addition, silicone rubber may be silicone rubber that is cross-linked with organo-polysiloxane having a relatively low molecular weight.

The suction mechanism 1 is obtained by molding the silicone raw material composition through an appropriate molding method according to a desired shape. For example, the suction mechanism can be molded by injection molding, extrusion molding, or cast molding.

Meanwhile, it is preferable that the suction mechanism 1 have transmittance of equal to or greater than 90%, and more preferably, equal to or greater than 92%. The transmittance of the suction mechanism 1 that is molded using polydimethyl siloxane is approximately 94%, and the transmittance of the suction mechanism 1 that is molded using polydiphenyl siloxane is approximately 92%. Even though the suction mechanism is used for a long period, the transmittance thereof is maintained. When the suction mechanism 1 is molded using a rigid silicone resin such as polydimethyl siloxane, the suction mechanism is not likely to expand and is not likely to deteriorate on the short wavelength side such as ultraviolet rays, and thus it is appropriate for maintaining an optical property.

In particular, when the suction mechanism 1 is made of silicone rubber, volatile components such as moisture or low molecular siloxane are likely to remain, and thus it is preferable to volatilize the volatile components.

According to the suction mechanism 1 mentioned above, it is possible to pull a specific portion (measurement object) of a living body into the suction mechanism 1 through the suction hole 5 by removing air in the suction mechanism 1 through the exhaust hole 4 so as to reduce the pressure in the suction mechanism 1 after bringing the suction hole 5 into contact with the measurement object. Therefore, the specific portion of the skin can be sampled through a simple procedure.

In addition, since the suction mechanism 1 has transmittance, the portion (the specific portion of the skin) of the skin which is suctioned into the suction mechanism 1 is irradiated with light, and thus it is possible to optically measure light generated by the portion of the skin being irradiated with light.

Furthermore, in order to perform the above-mentioned optical measurement, a portion of skin which is sampled has only to be irradiated with light. Thus, it is possible to simplify a procedure of confirming a state of the skin as compared with the diagnosis method disclosed in PTL 1.

In addition, a result of the above-mentioned optical measurement is obtained immediately after the sampled portion of the skin is irradiated with light. For this reason, it is possible to shorten the time required for the procedure of confirming the state of the skin, as compared with the diagnosis method disclosed in PTL 1.

Furthermore, in spite of a simple method of pulling a portion of skin into the suction mechanism 1, when the internal volume of the suction mechanism 1 is properly increased, the pulled portion of the skin can also include an epidermal layer or a dermic layer.

As described above, it is possible to sample skin for the purpose of simply confirming a state of the skin including at least the epidermal layer or the dermic layer.

Meanwhile, examples of the measurement object through the measuring device 100 can include an arm, a wrist, an earlobe, a fingertip, a palm, a cheek, the inner side of an upper arm, and the like of an examinee.

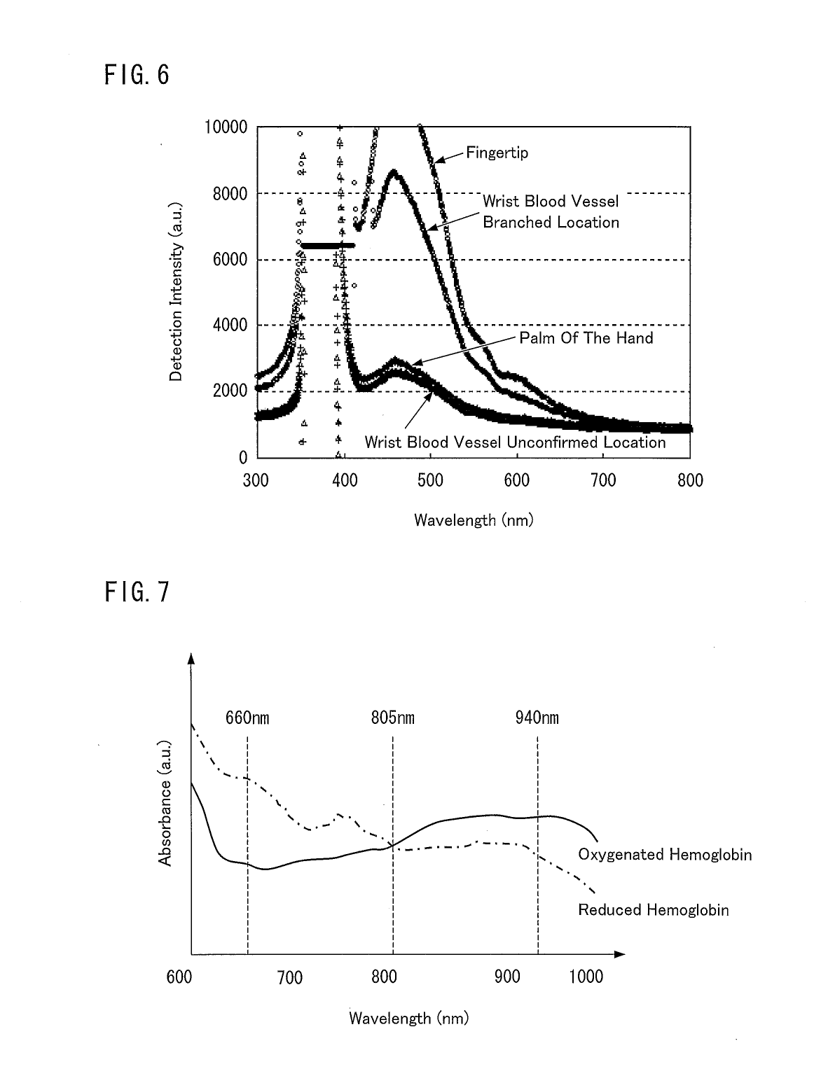

FIG. 6 illustrates a spectrum measurement result of fluorescence through AGEs from each location of the end of the hand (fingertip), a portion where blood vessels are branched (wrist blood vessel branched location) in blood vessels of the wrist, a portion where a blood vessel is not present in the wrist (wrist blood vessel unconfirmed location), and the palm of the hand (blood vessel unconfirmed location), among the measurement objects.

In FIG. 6, a horizontal axis represents a wavelength (nm) of fluorescence, and a vertical axis represents the intensity (a.u.) of fluorescence. For example, the intensity of fluorescence around a wavelength of 460 nm has a value of equal to or greater than 10,000 a.u. in the end of the hand (fingertip) and a value of approximately 9,000 a.u. in the portion where the blood vessels are branched (wrist blood vessel branched location), and thus a remarkable fluorescence spectrum is obtained. On the other hand, the fluorescence spectrum is obtained in the palm of the hand (blood vessel unconfirmed location) and the portion where a blood vessel is not present in the wrist (wrist blood vessel unconfirmed location), but a large numerical value is not obtained, as compared with the end of the hand (fingertip) and the portion where blood vessels are branched (wrist blood vessel branched location). It can be seen that the intensity of fluorescence varies to that extent in accordance with the branched location.

As described above, it can be seen that AGEs are particularly likely to be accumulated in the end of the hand (fingertip) and the portion where blood vessels are branched (wrist blood vessel branched location). In other words, it can be seen that exact data having a higher level of accuracy can be obtained by defining the location where the AGEs are likely to be accumulated, as a location to be measured.

Modified Example of Suction Mechanism 1

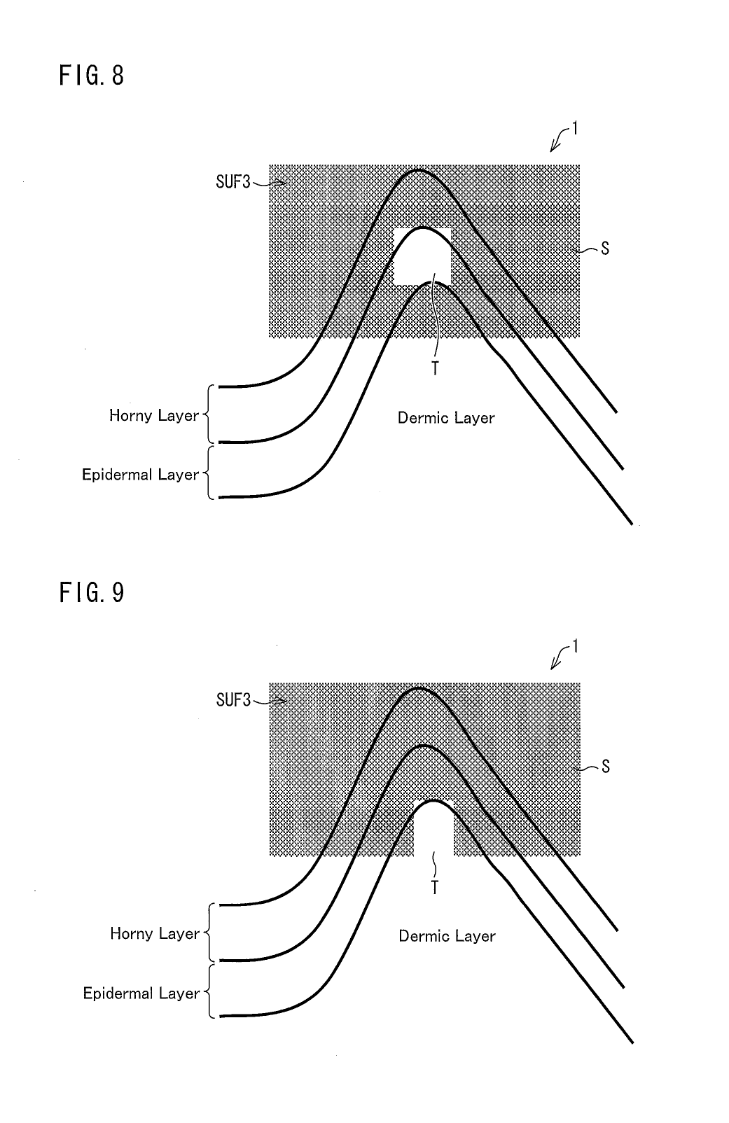

Next, a modified example of the above-mentioned suction mechanism 1 will be described with reference to FIGS. 8 and 9.

Incidentally, in the techniques disclosed in PTL 2 and PTL 3 mentioned above, transdermal fluorescence is detected as reflected light, and the device is also a large-scaled device. In addition, there is an additional problem in that not only transdermal fluorescence information obtained from a certain place but also a portion to be measured is not specified.

Consequently, as illustrated in FIGS. 8 and 9, in the suction mechanism 1, a portion of at least one surface other than the surface (light irradiation surface) SUF3 which is irradiated with light may be shielded from light (light shield portion S).

Meanwhile, as a method of providing the light shield portion S in the suction mechanism 1, a method can be considered of manufacturing a portion, from which fluorescence is not taken out, using light-shielding plastic, or a method of applying a light-shielding agent onto a surface of a transmissive material.

According to the above-mentioned configuration, it is possible to select and measure only fluorescence that is emitted from the portion (light shield portion T) which is not shielded from light, in the portion of at least one surface other than the light irradiation surface of the suction mechanism 1 which is irradiated with light. Therefore, it is possible to select and measure only fluorescence from a specific portion (for example, an epidermal layer or a dermic layer), in the portion of the skin which is suctioned into the suction mechanism 1.

For example, in FIG. 8, most parts other than the epidermal layer are covered by the light shield portion S. Therefore, it is possible to selectively detect only fluorescence emitted from the epidermal layer through the light shield portion T.

On the other hand, in FIG. 9, most parts other than the dermic layer are covered by the light shield portion S. Therefore, it is possible to selectively detect only fluorescence emitted from the dermic layer through the light shield portion T.

Meanwhile, the arrangement of the light shield portion S and the light shield portion T may be determined in advance according to an amount of skin to be suctioned when the internal pressure of the suction mechanism 1 is constant.

Light Source 2a

It is also possible to use not only a semiconductor device such as a light emitting diode (LED) or a laser diode (LD) but also a lamp light source, as the light source 2a.

Light having a wavelength between 315 nm to 400 nm which is a near-ultraviolet region and 315 nm to 600 nm which is a visible ray region is suitable for light emitted from the light source 2a.

Meanwhile, in this embodiment, the light has a wavelength that is equal to or greater than 230 nm and equal to or less than 365 nm which is a near-ultraviolet region, or a wavelength of 405 nm which is a blue-violet region.

A specific portion (for example, blood vessels) of a measurement object is irradiated with light having such a wavelength, and thus fluorescence is obtained from materials accumulated in blood vessel walls at an irradiation position.

In addition, a wavelength of light emitted from the light source 2a may be a wavelength within a range capable of detecting advanced glycation endproducts (AGEs).

AGEs can be detected based on the above-mentioned configuration. Meanwhile, since the intensity of AGEs-derived fluorescence increases in skin in which glycation progresses, it is possible to confirm the progress of glycation of the skin. Therefore, it is useful to realize the measuring device that detects AGEs.

There are approximately twenty types of AGEs currently known. Among these, there are several AGEs that emit fluorescence when irradiated with light. Table 1 shows an example thereof.

TABLE 1
Relationship Between Excitation Light Source and
Fluorescence Intensity of AGEs

| | Excitation (nm) | Emission (nm) |
|---|---|---|
| CLF collagen-linked | 370 | 440 |
| fluorescence | | |
| Pentosidine | 328 | 378 |
| | (After acid | (After acid |
| | hydrolysis: 335) | hydrolysis: 385) |
| Vesperlysines | 370 | 440 |

In Table 1, collagen-linked fluorescence (CLF) is fluorescence from AGEs combined with collagen, and is used as a general measure of the production of total AGEs and collagen cross-linking associated therewith.

Pentosidine and vesperlysine are representative examples of AGEs. Pentosidine has a structure in which an equimolar amount of lysine with respect to pentose is cross-linked with arginine, and is a fluorescent material that is stable after acid hydrolysis. In particular, it has been reported that pentosidine increases in diabetes onset and end-stage nephropathy. Vesperlysine has a structure in which AGE-modified bovine serum albumin (BSA) is acid-hydrolyzed and is then isolated as a main fluorescent material and two molecules of lysine are cross-linked with each other.

As seen from Table 1, a wavelength of 370 nm or a wavelength in the vicinity thereof is the most suitable for a wavelength of excitation light. However, a width between 315 nm to 400 nm which is an ultraviolet ray region and 315 nm to 600 nm which is a visible ray region is suitable for a width of excitation light that is adapted in accordance with types of AGEs.

Fluorescence is detected in this manner, and thus it is possible to non-invasively confirm the presence of AGEs from blood vessels.

Light Source 2b

Next, the light source (another light source) 2b is a near-infrared light source or an infrared light source for visualizing blood vessels. In addition, the light source 2b is preferably a light source capable of performing irradiation by switching between near-infrared light and infrared light. Examples of such a light source can include “multi-wavelength LED KED694M31D” manufactured by Kyosemi Corporation.

As a method of visualizing (detecting) blood vessels, it is also possible to measure AGEs by using a difference in absorbance between oxyhemoglobin combined with oxygen (oxygenated hemoglobin) and deoxyhemoglobin not combined with oxygen (reduced hemoglobin) in red and infrared regions and by specifying types of blood vessels (veins or arteries).

For example, a vein contains a large amount of reduced hemoglobin, while an artery contains a large amount of oxygenated hemoglobin. FIG. 7 illustrates a relationship between a wavelength and absorbance of oxygenated hemoglobin and reduced hemoglobin. A horizontal axis represents a wavelength (nm), and a vertical axis represents absorbance (a.u.). In the graph, reduced hemoglobin has a high absorbance on the short wavelength side and oxygenated hemoglobin has a high absorbance on the long wavelength side, with a wavelength of 805 nm being the boundary therebetween.

In other words, when light having a wavelength longer than 805 nm is irradiated, blood vessels (arteries) containing a large amount of oxygenated hemoglobin can be more clearly confirmed than blood vessels (veins) containing a small amount of oxygenated hemoglobin. Thereafter, when light having a wavelength shorter than 805 nm is irradiated, the blood vessels containing a large amount of oxygenated hemoglobin which are clearly viewed until then, that is, the blood vessels (arteries) containing a small amount of reduced hemoglobin become unclear, whereas the blood vessels containing a large amount of reduced hemoglobin, that is, the blood vessels (veins) containing a small amount of oxygenated hemoglobin become clear.

At this time, light having a long wavelength and light having a short wavelength with a wavelength of 805 nm being the boundary therebetween have been described as examples. However, it is theoretically possible to differentiate between reduced hemoglobin and oxygenated hemoglobin, in other words, between veins and arteries, and to visualize them by using a difference in relative absorbance between the long wavelength and the short wavelength. In other words, it is possible to identify the veins and the arteries by using a wavelength in which oxygenated hemoglobin has a higher absorbance than that of reduced hemoglobin and a wavelength in which oxygenated hemoglobin has a lower absorbance than that of reduced hemoglobin. Based on the data of FIG. 7, the use of light having a longer wavelength and a shorter wavelength with a wavelength of 805 nm being a boundary therebetween becomes suitable for differentiating between veins and arteries.

As described above, it is possible to differentiate between veins and arteries and to visualize them by using light having two or more types of wavelengths ranging from 600 nm to 1000 nm as the light source 2b. Referring to FIG. 7, it is particularly desirable to include a light source of a near-infrared region around 940 nm for detecting oxygenated hemoglobin and a light source of a red region around 660 nm for detecting reduced hemoglobin. These two types of wavelengths to be irradiated at the same location are rapidly switched with each other, and thus it is possible to compare blood vessel images thereof with each other and to identify the arteries and veins.

Meanwhile, a light source emitting near-infrared light and a light source emitting infrared light may be provided separately. For example, a near-infrared LED and a red LED are switched with each other and turned on, and thus it is possible to confirm whether or not a portion of skin which is sampled into the suction mechanism 1 includes blood vessels. The near-infrared LED is a light source that emits light having a wavelength of a near-infrared region around 945 nm (890 nm to 1010 nm). A skin surface is irradiated with near-infrared light, and it is possible to detect oxygenated hemoglobin and to visualize veins. The red LED is a light source that emits light having a wavelength of a red region around 660 nm (620 nm to 700 nm). The skin surface is irradiated with red light, and thus it is possible to detect reduced hemoglobin and to visualize arteries.

Detectors 3a and 3b

The detectors 3a and 3b are used to detect fluorescence emitted from a portion of skin, and include a single or plurality of light receiving elements. Meanwhile, the detectors 3a and 3b may include not only the light receiving element but also a spectroscope such as fluorescence spectrophotometer. Thus, it is possible to analyze detected data in more detail on the basis of dispersed fluorescence.

Examples of the light receiving element can include a semiconductor element such as a photo diode (PD), charge coupled devices (CCD), or a complementary metal-oxide-semiconductor (CMOS).

Since fluorescence emitted from the portion of the skin has a longer wavelength than excitation light, a detector capable of detecting light having a wavelength ranging from 350 nm to 500 nm may be used as the detector, from Table 1. However, with regard to fluorescence, since a wavelength that is detected by types of AGEs has a width, a detector capable of detecting a wavelength ranging from 320 nm to 900 nm can be used.

Meanwhile, in this embodiment, although another optical part is not present between the suction mechanism 1 and the detectors 3a and 3b, a single or plurality of optical members may be present between the suction mechanism 1 and the detectors 3a and 3b.

Examples of the optical part can include not only various optical members but also a light guide member such as an optical fiber.

Various optical members are members that change a state of fluorescence emitted from a portion of skin. Examples of the optical member can include a prism, a lens, a wavelength conversion element, an optical filter, a diffraction grating, a polarizing plate, a light path changing member, and the like. In addition, the “lens” is a member that adjusts a spot diameter of fluorescence. In addition, the “wavelength conversion element” is a member that converts fluorescence into light having a different wavelength. The “optical filter” is a member that blocks light having a wavelength in a predetermined wavelength range and transmits light having a wavelength in other than the range. The “light path changing member” is a member that changes a light path of a laser beam, for example, a mirror.

Meanwhile, since fluorescence generated inside the suction mechanism 1 spreads isotropically, the detectors 3a and 3b can be installed in an arbitrary position in the vicinity of the suction mechanism 1, except for a position at which the detector 3a or the detector 3b cannot be installed, due to the presence of the skin suctioned by the suction hole 5.

However, the fluorescence is often detected at a position at 90 degrees with respect to a propagation direction of excitation light, which is less influenced by reflected light, and thus it is preferable to detect the fluorescence from the vicinity of the surface SUF4 that is opposite to the suction hole 5 (the side coming into contact with the skin) in a similar manner to the detector 3b of FIG. 1. Besides, the fluorescence may be detected from the vicinity of the surface SUF5 that is not disturbed by the presence of the duct 6. In addition, the fluorescence may be detected from the vicinity of the surface SUF1 as long as it is not disturbed by the presence of the duct 6.

In addition, similarly to the detector 3a of FIG. 1, the detector may be installed in the vicinity of the surface (surface on the opposite side) SUF3 on the side opposite to the surface (surface on the side irradiated with light, light irradiation surface) SUF2 on the side irradiated with excitation light. Meanwhile, light detected by the detector 3a is not limited to fluorescence emitted from a portion of skin, and also includes transmitted light of excitation light, emitted from the light sources 2a and 2b, which passes through the portion of the skin.

Furthermore, the detector may be installed in the vicinity of the surface SUF2 as long as it is a position that does not disturb the irradiation of the excitation light emitted from the light sources 2a and 2b. In this case, light to be detected is not limited to fluorescence emitted from the portion of the skin, and also includes reflected light of the excitation light, emitted from the light sources 2a and 2b, which is reflected by the portion of the skin.

When the detector is installed in the vicinity of the surface SUF3, light to be detected by the detector is not limited to fluorescence emitted from the portion of the skin, and also includes reflected light of the excitation light, emitted from the light sources 2a and 2b, which is reflected by the portion of the skin. In this case, the detector may be formed to have a shape of a coaxial fiber capable of detecting the fluorescence and the reflected light using one fiber. In any case, the intensity of fluorescence which is received by the detectors 3a and 3b is measured, and thus it is possible to measure an amount of material (for example, AGEs) which is accumulated within a living body.

In addition, any one of the detectors 3a and 3b may be an imaging device that images a measurement object in order to visualize blood vessels included in a portion of skin. Examples of the imaging device can include a CCD camera or a CMOS camera in which light receiving elements are arranged in an array (or a matrix), but any of other imaging devices may be used.

The imaging device is installed on the outside of the suction mechanism 1 and images a measurement object. Meanwhile, an IR cut filter, which transmits visible rays and reflects infrared rays, may be installed in front of an imaging device in digital cameras being sold on the market, but a bandpass filter that transmits only light of a near-infrared region may be installed instead of the IR cut filter.

Pump 7

Incidentally, in the diagnosis method disclosed in PTL 1 mentioned above, although a state of the corneocytes (horny layer) can be confirmed, there is an additional problem that states of the epidermal layer and the dermic layer cannot be confirmed.

For example, elasticity of the skin is influenced not only by a state of the horny layer having a thickness of only 0.02 mm illustrated in FIG. 10(a) but also by a state of the epidermal layer (thickness of 0.07 mm to 0.2 mm), and furthermore, a state of the dermic layer (thickness of 2 mm). Equal to or greater than 70% of the dermic layer is formed of collagen fiber, but AGEs are cross-linked with the collagen fiber. A three-dimensional network structure of the collagen fiber collapses, and fibroblasts, hyaluronic acid, and the like are reduced, due to the crosslink through the AGEs. In addition, a structure of a basal layer at a boundary between the epidermal layer and the dermic layer collapses due to the crosslink through the AGEs, and thus the boundary between the dermic layer and the epidermal layer becomes unclear. As a result, the elasticity of the skin is decreased, and dullness of the skin progresses from the problem of a color tone of the AGEs.

In addition, as illustrated in FIG. 10(b), the horny layer repeats a turnover with a period of 14 days, while the epidermis and the dermis repeat a turnover with a period of 28 days and a period of 5 to 6 years, respectively. Therefore, it is obvious that health status of the epidermis and the dermis cannot be neglected in skin care.

In order to solve the above-mentioned additional problem, the measuring device 100 may include the pump 7 that decompresses the inside of the suction mechanism 1 through the exhaust hole 4 of the suction mechanism 1. Meanwhile, as illustrated in FIG. 1, the pump 7 is connected to the exhaust hole 4 through the duct 6.

Here, the pump 7 may be an electric pump or a manual pump, but “NMP05S” manufactured by KNF Japan Co., Ltd. or “Micro ring pump DSA-2-12BL” manufactured by AQUA Tech Co., Ltd. may be used as the pump 7.

According to the above-mentioned configuration, it is possible to decompress the inside of the suction mechanism 1 by using the pump 7 and to suction a portion of skin into the suction mechanism 1 through the suction hole 5.

In addition, it is possible to adjust an amount of the skin to be suctioned into the suction mechanism 1 by adjusting negative pressure of the inside of the suction mechanism 1 through the pump 7.

For example, according to the suction mechanism 1, it is possible to measure AGEs-derived fluorescence present in a horny layer, an epidermal layer, and/or a dermic layer. For this reason, the intensity of fluorescence to be detected is associated in advance with the amount of AGEs present in the horny layer, the epidermal layer, and/or the dermic layer, and thus it is also possible to specify the amount of AGEs present in the horny layer, the epidermal layer, and/or the dermic layer. In addition, according to the measuring device 100, it is also possible to know which part is glycosylated in the horny layer, the epidermal layer, and/or the dermic layer, and thus it is possible to use a skin sampling member at the scene of counseling such as confirmation of a cosmetic effect.

Incidentally, in the techniques disclosed in PTL 2 and PTL 3 mentioned above, transdermal fluorescence is detected as reflected light, and the device is also a large-scaled device. In addition, there is an additional problem in that not only transdermal fluorescence information obtained from a certain place but also a portion to be measured is not specified.

However, according to the measuring device 100, since it is possible to adjust an amount of skin to be sampled by adjusting the degree of decompression of the pump 7, the above-mentioned additional problem can also be simply solved.

In addition, in the techniques disclosed in PTL 2 and PTL 3 mentioned above, since the intensity of fluorescence is greatly different when blood vessels are present in a portion to be measured, there is also an additional problem that an obtained result is poor in quantitativity.

However, according to the measuring device 100, since it is possible to sample the skin without blood vessels present in a dermic layer by adjusting the degree of decompression of the pump 7, the above-mentioned additional problem can also be solved.

In addition, in the measuring device 100, the internal pressure (or volume) of the suction mechanism 1 through the decompression of the pump 7 may be at least variable from a first magnitude to a second magnitude or a third magnitude.

According to the above-mentioned configuration, the internal pressure (or volume) of the suction mechanism 1 may be at least variable from the first magnitude to the second magnitude or the third magnitude. Here, the first magnitude is such a magnitude that most of a portion of skin is constituted by a horny layer. In addition, the second magnitude is such a magnitude that most of a portion of skin is constituted by a horny layer and an epidermal layer. Furthermore, the third magnitude is such a magnitude that a portion of skin includes at least a dermic layer.

Therefore, when the internal pressure (or volume) of the suction mechanism 1 has the first magnitude, most of a portion of skin which is suctioned into the suction mechanism 1 can be constituted by a horny layer. In addition, when the internal pressure (or volume) of the suction mechanism 1 has the second magnitude, most of a portion of skin which is suctioned into the suction mechanism 1 can be constituted by a horny layer and an epidermal layer. Furthermore, when the internal pressure (or volume) of the suction mechanism 1 has the third magnitude, a portion of skin which is suctioned into the suction mechanism 1 can include at least a dermic layer.

Thus, for example, it is possible to measure AGEs-derived fluorescence present in the horny layer, the epidermal layer, and/or the dermic layer. For this reason, the intensity of fluorescence to be detected is associated in advance with an amount of AGEs present in the horny layer, the epidermal layer, and/or the dermic layer, and thus it is also possible to specify the amount of AGEs present in the horny layer, the epidermal layer, and/or the dermic layer. In addition, according to the above-mentioned configuration, it is also possible to know which part is glycosylated in the horny layer, the epidermal layer, and/or the dermic layer, and thus it is possible to use the measuring device at the scene of counseling such as confirmation of a cosmetic effect.

Next, a description will be given of a result obtained by performing an experiment for investigating the degree of negative pressure of the inside of the suction mechanism 1 by using a micropump.

In the experiment, three types of micropumps including (A) 0.45 (ml/min; milliliters/minute), (B) 6.4 (ml/min), and (C) 0.4 (1/min; liters/minute) were used in the experiment.

According to the micropump of (A) mentioned above, it was possible to confirm that most of a portion of skin is constituted by a horny layer and an epidermal layer (pressure having the second magnitude).

According to the micropump of (B) mentioned above, it was possible to confirm that negative pressure of −20 kPa (based on atmospheric pressure) is accomplished and the portion of the skin includes a dermic layer (pressure having the third magnitude).

According to the micropump of (C) mentioned above, it was possible to confirm that negative pressure of −50 kPa is accomplished.

From the above, it was possible to confirm that the internal pressure of the suction mechanism 1 can be variable from the first magnitude to the second magnitude or the third magnitude.

Control Unit 8

As illustrated in FIG. 1, the control unit 8 includes a pump control unit 81, a light source control unit 82, a detected data analysis unit 83, and a display control unit 84.

The pump control unit 81 controls the pump 7 so that the internal pressure (or volume) of the suction mechanism 1 can be maintained constant or that the internal pressure can be changed from at least the first magnitude to the third magnitude.

In addition, the light source control unit 82 controls the light sources 2a and 2b so that the light sources 2a and 2b can be turned on or turned off, that the intensity of light emitted from each light source can be adjusted, and that light emitted from the light source 2b can be switched from near red light to red light.

The detected data analysis unit 83 acquires detected data that is created by a signal detected by the detectors 3a and 3b being amplified by the signal conversion unit 10 and being A/D (digital/analog) converted, and outputs an analysis result thereof.

In addition, the detected data analysis unit 83 may specify the intensity of fluorescence emitted from an epidermal layer, on the basis of a difference in intensity between fluorescence detected when the internal pressure (or volume) of the suction mechanism 1 has the second magnitude and fluorescence detected when the internal pressure (or volume) of the suction mechanism 1 has the first magnitude.

Thus, the intensity of fluorescence emitted from the epidermal layer is associated in advance with a state of the epidermal layer (or the skin), and thus it is possible to confirm the state of the epidermal layer (or the skin).

In a reflective measuring device of the related art as described in the technique disclosed in PTL 2 or PTL 3, there is a problem that the reflection of excitation light may be superimposed on a fluorescence spectrum. Furthermore, in this reflective measuring device, when blood vessels are present in a measurement object, there is also a problem that fluorescence from AGEs accumulated in the blood vessels present in a lower portion of the measurement object may be superimposed on portions other than the skin.

However, according to the measuring device 100, since the difference in intensity between the fluorescence detected at the time of the second magnitude and the fluorescence detected at the time of the first magnitude is taken, it is possible to reduce the influence of reflected light of the light with which the portion of the skin is irradiated being superimposed on the fluorescence emitted from the epidermal layer.

Incidentally, melanin is a pigment (having a range in color from black to yellow) which is made within melanocytes (pigment cells) present in a portion of a basal layer of an epidermis illustrated in FIG. 10(a).

Usually, melanin does not remain within the melanocyte. The melanin is transferred to epidermis cells, rises up to a horny layer in the outermost surface of the skin by metabolism of the skin which is referred to as a turnover, and then becomes dirt together with an old horny layer and is stripped off. However, when a “freckle” occurs, the epidermis cells containing melanin remains in the basal layer as it is, or the melanocyte itself moves into the dermis as the case may be. In addition, there are various types of “freckles” such as a chloasma, a senile pigment freckle, or a birthmark, and it is known that they have different melanin distributions. In this manner, the melanin distribution in the skin is wide-ranging in scope not only up to melanocytes but also up to the epidermal layer and the dermic layer. This is also referred to as a trouble caused by a defect of keratinocyte present in the epidermis.

Here, the melanin contained in the epidermal layer affects a detection result of light generated by a portion of skin being irradiated with light.

However, according to the above-mentioned configuration of the measuring device 100, it is possible to remove information of a color tone (color difference information such as melanin, L*, a*, or b*) of the skin from collagen-derived information of the epidermal layer, and thus it is possible to more exactly analyze a state of the skin.

In addition, the detected data analysis unit 83 may specify the intensity of fluorescence emitted from the dermic layer, on the basis of a difference in intensity between the fluorescence detected when the internal pressure (or volume) of the suction mechanism 1 has the third magnitude and the fluorescence detected when the internal pressure (or volume) of the suction mechanism 1 has the first magnitude.

Thus, the intensity of fluorescence emitted from the dermic layer is associated in advance with a state of the dermic layer (or the skin), and thus it is possible to confirm the state of the dermic layer (or the skin).

In addition, according to the above-mentioned configuration, since the difference in intensity between the fluorescence detected at the time of the third magnitude and the fluorescence detected at the time of the first magnitude is taken, it is possible to reduce the influence of reflected light of the light with which the portion of the skin is irradiated being superimposed on the fluorescence emitted from the epidermal layer.

Next, the display control unit 84 receives an analysis result from the detected data analysis unit 83, creates an analysis result display image for presenting the analysis result to a user, sends the image to the display unit 11, and displays the analysis result display image on the display unit 11.

Meanwhile, information displayed as the analysis result display image includes an amount of AGEs present in a horny layer, an epidermal layer, and/or a dermic layer, a state of the skin which corresponds to the amount of AGEs, an image of visualized blood vessels (arteries or veins), and the like.

Recording Unit 9

Examples of various pieces of information that are recorded in the recording unit 9 can include not only an OS or control program for operating the measuring device 100, but also

(1) a value of a frequency of current or a PMW signal (pulse-width modulation signal) to be supplied to the light sources 2a and 2b,

(2) information (look-up table) indicating a relationship between the intensity of fluorescence detected by the detectors 3a and 3b and an amount of AGEs present in a horny layer, an epidermal layer, and/or a dermic layer,

(3) information (look-up table) indicating a relationship between the intensity of fluorescence detected by the detectors 3a and 3b and a state of the skin,

(4) data required for the generation of the analysis result display image, and the like.

In addition, the recording unit 9 may record an analysis result that is output by the detected data analysis unit 83.

In addition, a measuring method according to an embodiment of the present invention is a measuring method using the above-mentioned suction mechanism 1, and includes processes (1) to (3) below.

(1) a decompression process of decompressing the inside of the suction mechanism 1 through the exhaust hole 4.

(2) a light irradiation process of irradiating a portion of the skin, which is suctioned into the suction mechanism 1 through the suction hole 5 in the decompression process, with light.

(3) a light detection process of detecting light generated by the portion of the skin being irradiated with light in the light irradiation process.

According to the above-mentioned method, in the decompression process, air in the suction mechanism 1 is removed through the exhaust hole 4 so as to reduce the pressure in the suction mechanism 1. Therefore, it is possible to pull a portion of skin into the suction mechanism 1 by removing air in the suction mechanism 1 so as to reduce the pressure in the suction mechanism 1 after bringing the suction hole 5 into contact with the specific portion of the skin. Therefore, the specific portion of the skin can be sampled through a simple procedure.

In addition, in the light irradiation process, the portion of the skin, which is suctioned into the suction mechanism 1 through the suction hole 5 in the decompression process, is irradiated with light. Furthermore, in the light detection process, light generated by the portion of the skin being irradiated with light in the light irradiation process is detected. Therefore, the above-mentioned optical measurement can be performed. For this reason, it is possible to shorten the time required for the procedure of confirming the state of the skin, as compared with the diagnosis method disclosed in PTL 1.

From the above, it is possible to simply confirm the state of the skin including at least an epidermal layer or a dermic layer.

Here, in the related art, flexibility of the skin is evaluated by exciting a horny layer, which is extracted through a tape stripping method of sampling the horny layer using an adhesive tape, by ultraviolet rays and by evaluating fluorescence derived from a β sheet structure of keratin.

In addition, in the configuration disclosed in PTL 1 mentioned above, the horny layer is extracted from the skin by using an adhesive tape, the adhesive tape is melted by using an organic solvent over half or more days, a sample is prepared by using the extracted horny layer as a microscope observation sample, and the measurement of fluorescence is performed using a fluorescence spectrophotometer in the prepared sample. In other words, there is a problem that a result is not obtained if one or more days have not elapsed after the examinee feels a desire to confirm his or her skin state. When cosmetic counseling is performed, in a case where opinions cannot be exchanged with each other on the basis of his or her skin data at the scene, there are problems of complication such as counseling being performed at a later date and inefficiency, after the extraction of skin tissues. In addition, actually, the elasticity of the skin is influenced not only by a state of the horny layer having a thickness of only 0.02 mm but also by a state of the epidermal layer (thickness of 0.07 mm to 0.2 mm), and furthermore, a state of the dermic layer (thickness of 2 mm). Equal to or greater than 70% of the dermic layer is formed of collagen fiber, but AGEs are cross-linked with the collagen fiber. A three-dimensional network structure of the collagen fiber collapses, and fibroblast, hyaluronic acid, and the like are reduced, due to the crosslink through the AGEs. In addition, a structure of a basal layer at a boundary between the epidermal layer and the dermic layer collapses, and thus the boundary between the dermic layer and the epidermal layer becomes unclear. As a result, the elasticity of the skin is decreased, and dullness of the skin progresses from the problem of a color tone of the AGEs.

In addition, the horny layer repeats a turnover with a period of 14 days, while the epidermis and the dermis repeat a turnover with a period of 28 days and a period of 5 to 6 years, respectively. Therefore, it is obvious that health status of the epidermis and the dermis cannot be neglected in skin care.

According to the measuring device 100 (or a measuring device 200 to be described below) of this embodiment, the above-mentioned problems can be solved.

2. Measuring Device 200

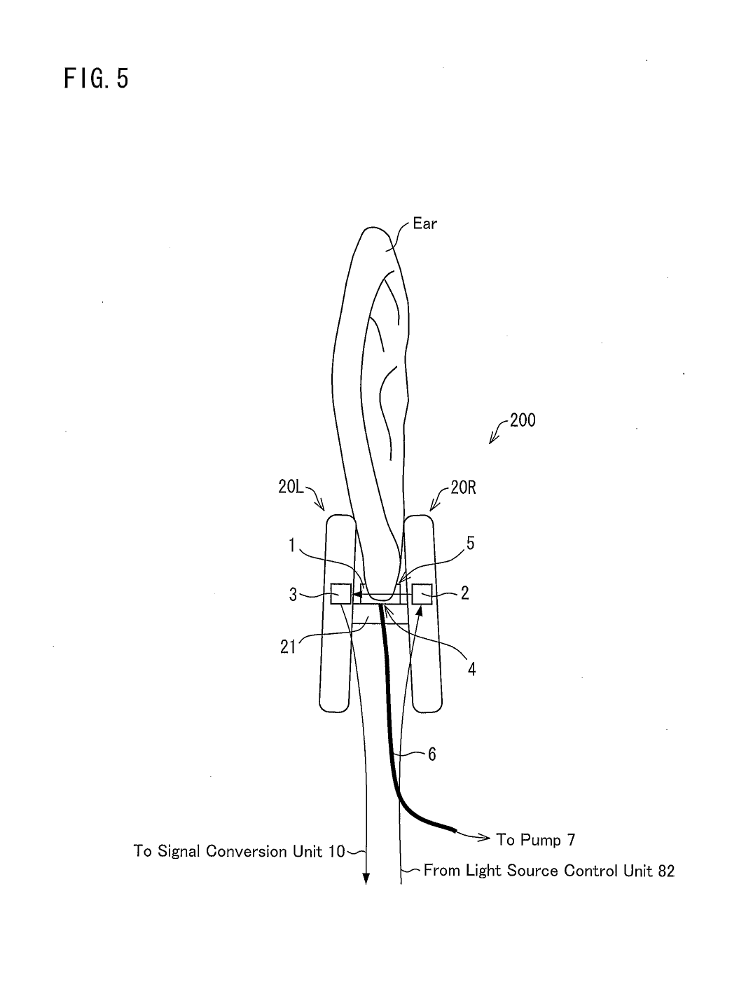

First, the whole configuration of the measuring device 200 (ear sensor type) according to another embodiment of the present invention will be described with reference to FIG. 5. FIG. 5 is a diagram illustrating the whole configuration of the measuring device 200.

The measuring device 200 is different from the measuring device 100, in that

(1) the respective number of light sources 2 and detectors (light detection units) 3 is one, and

(2) brackets (clips) 20L and 20R and a hinge (clip) 21 are included therein.

Meanwhile, other configurations are the same as the measuring device 100, and thus a description thereof will not be repeated.

Light Source 2 and Detector 3

The light source 2 has the same function as any of the light source 2a or the light source 2b of the measuring device 100.

In addition, the detector 3 has the same function as the detector 3a or the detector 3b of the measuring device 100.

Brackets 20L and 20R and Hinge 21

As illustrated in FIG. 5, the measuring device 200 includes the brackets (clips) 20L and 20R for pinching a portion of an earlobe therebetween, and the hinge (clip) 21.

In addition, as illustrated in FIG. 5, the suction mechanism 1 is provided in a position capable of pulling the portion of the earlobe, which is pinched between the brackets 20L and 20R, through the suction hole 5. Meanwhile, the hinge 21 includes a spring for causing the brackets 20L and 20R to function as clips.

In addition, as illustrated in FIG. 5, the suction hole 5 of the suction mechanism 1 is provided in a location (the upper side in FIG. 5) which is opposite to the exhaust hole 4 (the lower side in FIG. 5) in a manner similar to that illustrated in FIG. 4.

According to the above-mentioned configuration, it is possible to perform the above-mentioned optical measurement by irradiating the portion of the earlobe which is suctioned into the suction mechanism 1 with light.

For example, in an earlobe, cosmetics are not necessarily required to be removed at the time of measurement of fluorescence. If the cosmetics are removed, the earlobe can be used without imposing a large burden on a user. In addition, the earlobe has a small number of blood vessels and has a small amount of fluorescence as a background through AGEs accumulated in blood vessel walls, and thus a more exact measurement can be performed. In addition, since the skin of the earlobe is extremely thinner than other portions, it is possible to confirm states of a horny layer, an epidermal layer, and/or a dermic layer without having to change the internal volume of the suction mechanism 1.

3. Conclusion

As described above, according to a measuring method using the measuring device 100 or 200 and the suction mechanism 1, it is possible to simply confirm a state of skin including at least an epidermal layer or a dermic layer.

In addition, an amount of skin to be sampled in the suction mechanism 1 is controlled, and thus it is possible to quantify living body information based on information of the skin in a depth direction. The detection of a fluorescent material within the living body which indicates different behaviors in a portion to be measured or at a measurement location is obtained as intensity information, and thus it is possible to visualize the degree of aging of the skin based on the information.

In addition, according to the measuring devices 100 and 200, it is possible to monitor a glycation state of the skin, and to monitor the degree of aging of the skin by using fluorescence emitted from AGEs accumulated in the epidermal layer and/or the dermic layer of the skin.

Furthermore, a portion of skin is sampled by the suction mechanism 1, and thus it is possible to know which layer the fluorescence is obtained from in the skin, to monitor a health status of the skin, and to rapidly and easily confirm effects and efficacy of anti-glycation cosmetics, from the amount of sampled portion.

Therefore, according to the measuring devices 100 and 200, it is also possible to visualize the confirmation of effects of the anti-aging cosmetics through the measurement of aging of the skin due to glycation, which cannot be realized in a measuring device of the related art.

Finally, each block of the measuring devices 100 and 200, in particular, the control unit 8, may be realized in a hardware manner by a logic circuit formed on an integrated circuit (IC chip), or may be realized in a software manner by using a central processing unit (CPU).

In the latter case, the measuring devices 100 and 200 include a CPU that executes a command of a program for implementing each function, a read only memory (ROM) that stores the program, a random access memory (RAM) that develops the program, and a storage device (recording medium; for example, the recording unit 9) such as a memory, which stores the program and various pieces of data. In addition, an object of the present invention can also be accomplished by supplying a recording medium, which records program codes (executable format program, intermediate code program, and source program) of a control program of the measuring devices 100 and 200 which are software for implementing the above-mentioned function so as to be readable by a computer, to the measuring devices 100 and 200 and by causing the computer (or CPU or MPU) to read and execute the program codes that are recorded in the recording medium.

For example, tapes such as a magnetic tape or a cassette tape, disks including a magnetic disk, such as a floppy (registered trademark) disk or a hard disk, or an optical disc such as a CD-ROM, an MO, an MD, a DVD, or a CD-R, cards such as an IC card (including a memory card) or an optical card, semiconductor memories such as a mask ROM, an EPROM, an EEPROM, or a flash ROM, or logic circuits such as a programmable logic device (PLD) or a field programmable gate array (FPGA) can be used as the recording medium.

In addition, the measuring devices 100 and 200 may be configured so as to be connected to a communication network, and the program codes may be supplied through the communication network. The communication network may be a network capable of transmitting the program code, and is not particularly limited. For example, the Internet, an intranet, an extranet, a LAN, an ISDN, a VAN, a CATV communication network, a virtual private network, a telephone network, a moving body communications network, a satellite communications network, or the like can be used. In addition, a transmission medium constituting the communication network may be a medium capable of transmitting the program code, and is not limited to a medium having a specific configuration or a specific type of medium. For example, a wired transmission medium such as IEEE1394, a USB, a power-line carrier, a cable TV line, a telephone line, or an asymmetric digital subscriber line (ADSL), or a wireless transmission medium, e.g., infrared rays such as IrDA or a remote controller, Bluetooth (registered trademark), IEEE802.11 wireless, a high data rate (HDR), near field communication (NFC), digital living network alliance (DLNA), a mobile phone network, a satellite line, or a terrestrial digital network can be used.

In addition, the measuring device of the present invention can also be expressed as follows.

That is, the measuring device of the present invention may include a sampling mechanism (skin sampling member) that samples a portion of skin, an excitation light irradiation unit that irradiates the portion (location, a measurement object) of the skin, which is sampled by the sampling mechanism, with excitation light, and a light receiving unit that receives fluorescence generated by a living body being irradiated with the excitation light.

According to the above-mentioned configuration, an amount of skin to be sampled is controlled, and thus it is possible to quantify living body information based on information of the skin in a depth direction. The detection of a fluorescent material within the living body which indicates different behaviors in a portion to be measured or at a measurement location is obtained as intensity information, and thus an effect such as the visualization of the degree of aging of the skin based on the information is exhibited.

According to the above-mentioned configuration, it is possible to monitor information of skin based on presence locations (location information) of a horny layer, an epidermal layer, and/or a dermic layer of the skin in a cross-sectional direction of the skin, in accordance with an amount of skin to be sampled. The information of fluorescence includes the intensity of fluorescence, information of a detected wavelength, and material-derived physical property information such as a half-value width thereof. The above-mentioned configuration is used, and thus it is possible to reduce the influence of reflected light of excitation light being superimposed on AGEs-derived fluorescence with respect to a measurement object. In addition, blood vessels are present in the dermic layer, and it is possible to exclude AGEs-derived fluorescence accumulated in blood vessel walls. For example, in an arm, a wrist, an earlobe, a fingertip, a palm, a cheek, and the like, blood vessels are present in detection locations thereof, and thus it is confirmed from an experiment that the intensity of fluorescence becomes higher than at a location not including blood vessels. In addition, it is also possible to confirm the presence of blood vessels by confirming an image of infrared rays.

In addition, in the measuring device of the present invention, the sampling mechanism may include a structure of which the size is variable, in order to select (sample) an intended layer. More specifically, in the measuring device of the present invention, the sampling mechanism may be configured to be capable of changing lengths (sizes) of sides of three surfaces including a surface to be irradiated with the excitation light, a surface through which the excitation light passes, and a surface in which fluorescence is detected, and two lateral surfaces other than a surface for pulling the skin.

Based on the above-mentioned configuration, it is possible to measure an amount of AGEs, which are present in a horny layer, an epidermal layer, and/or a dermic layer of skin along a cross sectional direction of the skin, at approximately the same location.

In addition, in the measuring device of the present invention, the sampling structure may use a clip-type measuring mechanism, particularly, in an earlobe. More specifically, the measuring device of the present invention may have a sensing mechanism including an excitation light irradiation unit that clips, particularly, a portion of the earlobe and irradiates the portion with excitation light, and a light receiving unit that receives fluorescence generated by a living body being irradiated with the excitation light, in the sampling mechanism.

In particular, in an earlobe, cosmetics are not necessarily required to be removed at the time of measurement of fluorescence. If the cosmetics are removed, the earlobe can be used without imposing a large burden on a user. In addition, there is an advantage in view of measurement in that the earlobe has a small number of blood vessels and has a small amount of fluorescence as a background through AGEs accumulated in blood vessel walls. There is also an advantage in view of measurement in that the skin of the earlobe is extremely thinner than other portions. Excitation light from a light-emitting device may be connected to one clip using optical fibers, and optical fibers connected to the light receiving unit, which receives fluorescence generated by a living body being irradiated with the excitation light irradiation unit, may be connected to the other clip.

In addition, in the measuring device of the present invention, the excitation light may have an appropriate wavelength range in order to measure advanced glycation endproducts.

Based on the above-mentioned configuration, AGEs from a specific location of skin can be measured. The inventor of this application has newly found that fluorescence intensity of AGEs increases in skin in which glycation of the skin progresses. For this reason, it is useful to realize the measuring device that measures AGEs, as a skin sensor.

According to the above-described measuring device, it is possible to monitor a glycation state of the skin, and to monitor the degree of aging of the skin by using fluorescence emitted from glycation materials (AGEs) which are accumulated in the epidermal layer and/or the dermic layer of the skin. In addition, a portion of skin is sampled, and thus it is possible to know which layer the fluorescence is obtained from in the skin, from the amount of sampled portion. Therefore, it is also possible to monitor a health status of the skin and to rapidly and easily confirm effects and efficacy of anti-glycation cosmetics.

4. Another Expression of the Present Invention

The present invention can also be expressed as follows.

That is, in the skin sampling member of the present invention, the housing may include an elastic portion between a surface on the side at least irradiated with light and a surface on the side opposite thereto, and the whole housing may be formed of an elastic material.

According to the above-mentioned configuration, a distance between the side of the housing which is at least irradiated with light and the side opposite thereto becomes variable due to the presence of the elastic portion. In other words, the interval volume of the housing becomes variable.

Incidentally, in the techniques disclosed in PTL 2 and PTL 3 mentioned above, transdermal fluorescence (skin) is detected as reflected light, and the device is also a large-scaled device. In addition, there is an additional problem in that not only transdermal fluorescence information obtained from a certain place but also a portion to be measured is not specified.

Consequently, in the skin sampling member of the present invention, in order to solve such an additional problem, a portion of at least one surface other than a light irradiation surface of the housing which is irradiated with light may be shielded from light, in addition to the above-mentioned configuration.

According to the above-mentioned configuration, it is possible to select and measure only fluorescence that is emitted from the portion which is not shielded from light, in the portion of at least one surface other than the light irradiation surface of the housing which is irradiated with light. Therefore, it is possible to select and measure only fluorescence from a specific portion (for example, an epidermal layer or a dermic layer), in the portion of the skin which is suctioned into the housing.

In addition, the measuring device of the present invention may include the skin sampling member, a light source that irradiates a portion of skin, which is suctioned into the housing through the suction hole, with light, and a light detection unit that detects light generated by the portion of the skin being irradiated with light.

According to the above-mentioned configuration, it is possible to realize the measuring device that irradiates a portion of skin which is suctioned into the housing with light by the light source and detects light generated by the portion of the skin being irradiated with light by the light detection unit.

Meanwhile, a detection result (physical property information or physical quantities) of the light detection unit includes various pieces of physical property information, such as the intensity of detected light, a half-value width thereof, a wavelength of detected light, the reflectivity of the skin, or the transmittance of the skin, which are derived from a material contained in the portion of the skin.

Incidentally, in the diagnosis method disclosed in PTL 1 mentioned above, although it is possible to confirm a state of the corneocytes (horny layer), there is an additional problem that it is not possible to confirm states or a state of the epidermal layer and/or the dermic layer.

For example, elasticity of the skin is influenced not only by a state of the horny layer having a thickness of only 0.02 mm illustrated in FIG. 10(a) but also by a state of the epidermal layer (thickness of 0.07 mm to 0.2 mm), and furthermore, a state of the dermic layer (thickness of 2 mm). Equal to or greater than 70% of the dermic layer is formed of collagen fiber, but advanced glycation endproducts (AGEs) are cross-linked with the collagen fiber. A three-dimensional network structure of the collagen fiber collapses, and fibroblasts, hyaluronic acid, and the like are reduced, due to the crosslink through the AGEs. In addition, a structure of a basal layer at a boundary between the epidermal layer and the dermic layer collapses due to the crosslink through the AGEs, and thus the boundary between the dermic layer and the epidermal layer becomes unclear. As a result, the elasticity of the skin is decreased, and dullness of the skin progresses from the problem of a color tone of the AGEs.

In addition, as illustrated in FIG. 10(b), the horny layer repeats a turnover with a period of 14 days, while the epidermis and the dermis repeat a turnover with a period of 28 days and a period of 5 to 6 years, respectively. Therefore, it is obvious that health status of the epidermis and the dermis cannot be neglected in skin care.

In addition, in order to solve the above-mentioned additional problem, the measuring device of the present invention may include a pump that decompresses the inside of the housing through the exhaust hole (of the skin sampling member).

According to the above-mentioned configuration, it is possible to decompress the inside of the housing by using the pump and to suction a portion of skin into the housing through a suction hole.

In addition, it is possible to adjust an amount of the skin to be suctioned into the housing by adjusting negative pressure of the inside of the housing through the pump.

For example, according to the skin sampling member of the present invention, it is possible to measure AGEs-derived fluorescence present in the horny layer, the epidermal layer, and/or the dermic layer. For this reason, the intensity of fluorescence to be detected is associated in advance with an amount of AGEs present in the horny layer, the epidermal layer, and/or the dermic layer, and thus it is also possible to specify the amount of AGEs present in the horny layer, the epidermal layer, and/or the dermic layer. In addition, according to the above-mentioned configuration, it is also possible to know which portion is glycosylated in the horny layer, the epidermal layer, and/or the dermic layer, and thus it is possible to use the skin sampling member at the scene of counseling such as confirmation of a cosmetic effect.

Incidentally, as described above, in the techniques disclosed in PTL 2 and PTL 3 mentioned above, transdermal fluorescence is detected as reflected light, and the device is also a large-scaled device. In addition, there is an additional problem in that not only transdermal fluorescence information obtained from a certain place but also a portion to be measured is not specified.

However, according to the above-mentioned measuring device of the present invention, since it is possible to adjust an amount of skin to be sampled by adjusting the degree of decompression of the pump, the above-mentioned additional problem can also be simply solved.

In addition, in the techniques disclosed in PTL 2 and PTL 3 mentioned above, since the intensity of fluorescence is greatly different when blood vessels are present in a portion to be measured, there is also an additional problem that an obtained result is poor in quantitativity.

However, according to the above-mentioned measuring device of the present invention, since it is possible to sample the skin without blood vessels present in a dermic layer by adjusting the degree of decompression of the pump, the above-mentioned additional problem can also be solved.

In addition, the measuring method of the present invention is a measuring method using the above-mentioned skin sampling member, and may include a decompression process of decompressing the inside of the housing through the exhaust hole, a light irradiation process of irradiating a portion of skin, which is suctioned into the housing through the suction hole in the decompression process, with light, and a light detection process of detecting light generated by the portion of the skin being irradiated with light in the light irradiation process.

According to the above-mentioned method, in the decompression process, air in the housing is removed through the exhaust hole so as to reduce the pressure in the housing. Therefore, it is possible to pull a portion of skin into the housing by removing air in the housing so as to reduce the pressure in the housing after bringing the suction hole into close with the specific portion of the skin. Therefore, the specific portion of the skin can be sampled through a simple procedure.

In addition, in the light irradiation process, the portion of the skin, which is suctioned into the housing through the suction hole in the decompression process, is irradiated with light. Furthermore, in the light detection process, light generated by the portion of the skin being irradiated with light in the light detection process is detected. Therefore, the above-mentioned optical measurement can be performed. For this reason, it is possible to shorten the time required for the procedure of confirming the state of the skin, as compared with the diagnosis method disclosed in PTL 1.

From the above, it is possible to simply confirm the state of the skin including at least an epidermal layer or a dermic layer.

In addition, in the measuring device of the present invention, the internal pressure of the housing may be at least variable from such a first magnitude that most of a portion of the skin is constituted by a horny layer to such a second magnitude that most of a portion of the skin is constituted by a horny layer and an epidermal layer.

According to the above-mentioned configuration, the internal pressure of the housing may be at least variable from the first magnitude to the second magnitude. Here, the first magnitude is such a magnitude that most of a portion of the skin is constituted by a horny layer, and the second magnitude is such a magnitude that most of a portion of the skin is constituted by a horny layer and an epidermal layer.

Therefore, when the internal pressure of the housing has the first magnitude, most of a portion of the skin which is suctioned into the housing can be constituted by a horny layer. In addition, when the internal pressure of the housing has the second magnitude, most of a portion of the skin which is suctioned into the housing can be constituted by a horny layer and an epidermal layer.

Thus, for example, it is possible to measure AGEs-derived fluorescence present in the horny layer and/or the epidermal layer. For this reason, the intensity of fluorescence to be detected is associated in advance with an amount of AGEs present in the horny layer and/or the epidermal layer, and thus it is also possible to specify the amount of AGEs present in the horny layer and/or the epidermal layer.

In addition, the inventor of this application has newly found that the intensity of fluorescence of AGEs increases in skin with advanced glycation. Therefore, according to the above-mentioned configuration, it is also possible to know which portion is glycosylated in the horny layer and/or the epidermal layer, and thus it is possible to use the measuring device at the scene of counseling such as confirmation of a cosmetic effect.

In addition, the measuring device of the present invention may include a detected data analysis unit that specifies the intensity of fluorescence emitted from the epidermal layer, on the basis of a difference in intensity between fluorescence detected by the light detection unit when the internal pressure of the housing has the second magnitude and fluorescence detected by the light detection unit when the internal pressure of the housing has the first magnitude.

According to the above-mentioned configuration, the detected data analysis unit specifies the intensity of fluorescence emitted from the epidermal layer, on the basis of a difference in intensity between fluorescence detected when the internal pressure of the housing has the second magnitude and fluorescence detected when the internal pressure of the housing has the first magnitude.

Therefore, the intensity of fluorescence emitted from the epidermal layer is associated in advance with a state of the epidermal layer (or the skin), and thus it is possible to confirm the state of the epidermal layer (or the skin).

In a reflective measuring device of the related art such as the technique disclosed in PTL 2 or PTL 3, there is a problem that the reflection of excitation light may be superimposed on a fluorescence spectrum. Furthermore, in this reflective measuring device, when blood vessels are present in a measurement object, there is also a problem that fluorescence from AGEs accumulated in the blood vessels present in a lower portion of the measurement object may be superimposed on portions other than the skin.

However, according to the above-mentioned configuration, since the difference in intensity between the fluorescence detected at the time of the second magnitude and the fluorescence detected at the time of the first magnitude is taken, it is possible to reduce the influence of reflected light of light with which the portion of the skin is irradiated being superimposed on the fluorescence emitted from the epidermal layer.

Incidentally, melanin is a pigment (having a range in color from black to yellow) which is made within a melanocyte (pigment cell) present in a portion of a basal layer of an epidermis illustrated in FIG. 10(a).

Usually, melanin does not remain within melanocytes. The melanin is transferred to an epidermis cell, rises up to a horny layer in the outermost surface of the skin by metabolism of the skin which is referred to as a turnover, and then becomes dirt together with an old horny layer and is stripped off. However, when a “freckle” occurs, the epidermis cell containing melanin remains in the basal layer as it is, or the melanocyte itself moves into the dermis as the case may be. This is also referred to as a trouble caused by a defect of keratinocytes present in the epidermis. In addition, there are various types of “freckles” such as a chloasma, a senile pigment freckle, or a birthmark, and it is known that they have different melanin distributions. In this manner, the melanin distribution in the skin is wide-ranging in scope not only up to melanocytes but also up to the epidermal layer and the dermic layer.

Here, the melanin contained in the epidermal layer affects a detection result of light generated by a portion of skin being irradiated with light.

However, according to the above-mentioned configuration of the measuring device, it is possible to remove information of a color tone (color difference information such as melanin, L*, a*, or b*) of the skin from collagen-derived information of the epidermal layer, and thus it is possible to more exactly analyze a state of the skin.

In addition, in the measuring device of the present invention, the internal pressure of the housing may be at least variable from such a first magnitude that most of a portion of skin is constituted by a horny layer to such a third magnitude that the portion of the skin includes at least a dermic layer.

According to the above-mentioned configuration, the internal pressure of the housing may be at least variable from the first magnitude to the third magnitude. Here, the first magnitude and the second magnitude are as described above, and the third magnitude is such a magnitude that the portion of the skin includes at least a dermic layer.

Therefore, when the internal pressure of the housing has the first magnitude and the second magnitude, a configuration is given as described above. However, when the internal pressure of the housing has the third magnitude, a configuration can be given such that the portion of the skin which is suctioned into the housing includes at least a dermic layer.

Thus, for example, it is possible to measure AGEs-derived fluorescence present in the horny layer, the epidermal layer, and/or the dermic layer. For this reason, the intensity of fluorescence to be detected is associated in advance with an amount of AGEs present in the horny layer, the epidermal layer, and/or the dermic layer, and thus it is also possible to specify the amount of AGEs present in the horny layer, the epidermal layer, and/or the dermic layer. In addition, according to the above-mentioned configuration, it is also possible to know which portion is glycosylated in the horny layer, the epidermal layer, and/or the dermic layer, and thus it is possible to use the measuring device at the scene of counseling such as confirmation of a cosmetic effect.

In addition, the measuring device of the present invention may include a detected data analysis unit that specifies the intensity of fluorescence emitted from the dermic layer, on the basis of a difference in intensity between fluorescence detected by the light detection unit when the internal pressure of the housing has the third magnitude and fluorescence detected by the light detection unit when the internal pressure of the housing has the first magnitude.

According to the above-mentioned configuration, the detected data analysis unit specifies the intensity of fluorescence emitted from the dermic layer, on the basis of a difference in intensity between fluorescence detected when the internal pressure of the housing has the third magnitude and fluorescence detected when the internal pressure of the housing has the first magnitude.

Therefore, the intensity of fluorescence emitted from the dermic layer is associated in advance with a state of the dermic layer (or the skin), and thus it is possible to confirm the state of the dermic layer (or the skin).

In addition, according to the above-mentioned configuration, since the difference in intensity between the fluorescence detected at the time of the third magnitude and the fluorescence detected at the time of the first magnitude is taken, it is possible to reduce the influence of reflected light of light with which the portion of the skin is irradiated being superimposed on the fluorescence emitted from the dermic layer.

In addition, in the measuring device of the present invention, a wavelength of light emitted from the light source may be a wavelength within a range capable of detecting advanced glycation endproducts (AGEs).

AGEs can be detected based on the above-mentioned configuration. Meanwhile, as described above, since the intensity of AGEs-derived fluorescence increases in skin with advanced glycation, it is possible to confirm the progress of glycation of the skin. Therefore, it is useful to realize the measuring device for detecting AGEs.

In addition, the measuring device of the present invention may further include another light source that irradiates a portion of skin with near-infrared light or infrared light.

According to the above-mentioned configuration, it is possible to detect oxygenated hemoglobin and to detect veins by irradiating a skin surface with near-infrared light from another light source.

In addition, it is possible to detect reduced hemoglobin and to detect arteries by irradiating a skin surface with red light from another light source.

In addition, the measuring device of the present invention includes clips for pinching a portion of an earlobe therebetween, and the skin sampling member may be provided in a position capable of pulling the portion of the earlobe, which is pinched between the clips, through the suction hole.

According to the above-mentioned configuration, it is possible to perform an optical measurement by irradiating the portion of the earlobe, which is suctioned into the housing, with light.

For example, in an earlobe, cosmetics are not necessarily required to be removed at the time of measurement of fluorescence. If the cosmetics are removed, the earlobe can be used without imposing a large burden on a user. In addition, the earlobe has a small number of blood vessels and has a small amount of fluorescence as a background through AGEs accumulated in blood vessel walls, and thus a more exact measurement can be performed. In addition, since the skin of the earlobe is extremely thinner than other portions, it is possible to confirm states of a horny layer, an epidermal layer, and/or a dermic layer without having to change the internal volume of the housing.

Addition

The present invention is not limited to the above-mentioned embodiments, and various modifications can be made within scopes described in the claims. In addition, any other embodiments achieved by the appropriate combination of technical means disclosed in different embodiments are also included in the technical scope of the present invention.

### INDUSTRIAL APPLICABILITY

A sampling member, a measuring device, and a measuring method of the present invention can be applied to a monitoring device capable of monitoring a glycation state of skin, which cannot be achieved by a technique of the related art. In addition, they can also be applied to a monitoring device, for monitoring health status of skin, which anyone can use easily with a high level of accuracy. Therefore, in the confirmation of effects and efficacy of anti-glycation cosmetics, promotion of the acquirement of evidence is expected, and an application as a skin care monitoring device is expected.

### REFERENCE SIGNS LIST

1 SUCTION MECHANISM (SKIN SAMPLING MEMBER, HOUSING)
2, 2a LIGHT SOURCE
2b LIGHT SOURCE (ANOTHER LIGHT SOURCE)
3, 3a, 3b DETECTOR (LIGHT DETECTION UNIT)
4 EXHAUST HOLE
5 SUCTION HOLE
6 DUCT
7 PUMP
8 CONTROL UNIT
9 RECORDING UNIT
10 SIGNAL CONVERSION UNIT
11 DISPLAY UNIT
20L, 20R BRACKET (CLIP)
21 HINGE (CLIP)
81 PUMP CONTROL UNIT
82 LIGHT SOURCE CONTROL UNIT
83 DETECTED DATA ANALYSIS UNIT
84 DISPLAY CONTROL UNIT
100, 200 MEASURING DEVICE
T LIGHT SHIELD PORTION
S LIGHT SHIELD PORTION
SUF1 SURFACE
SUF2 SURFACE (SURFACE ON THE SIDE IRRADIATED WITH LIGHT, LIGHT IRRADIATION SURFACE)
SUF3 SURFACE (SURFACE ON OPPOSITE SIDE)
SUF4, SUF5 SURFACE

1-13. (canceled)

14: A measuring device comprising:
a skin sampling member including a housing that is formed of a transmissive material; a suction hole, provided in the housing, which suctions skin; and an exhaust hole, provided in the housing, which decompresses the inside of the housing;
a light source that irradiates a portion of skin, which is suctioned into the housing through the suction hole, with light; and
a light detection unit that detects fluorescence generated by the portion of the skin being irradiated with excitation light.

15: The measuring device according to claim 14, wherein the housing has an elastic member at least between a surface to be irradiated with light and a surface opposed to the surface to be irradiated with the light.

16: The measuring device according to claim 14, wherein part of at least one surface other than the surface to be irradiated blocks the light from entering the housing.

17: The measuring device according to claim 14, further comprising a pump that removes air in the housing through the exhaust hole so as to reduce pressure in the housing.

18: The measuring device according to claim 17, wherein the pressure in the housing is variable at least from a first pressure under which the section of the skin pulled into the housing substantially includes a horny layer alone, to a second pressure under which the section of the skin pulled into the housing substantially includes a horny layer and an epidermal layer alone.

19: The measuring device according to claim 17, wherein the pressure in the housing is variable at least from a first pressure under which the section of the skin pulled into the housing substantially includes a horny layer alone, to a third pressure under which the section of the skin pulled into the housing includes a dermic layer.

20: The measuring device according to claim 18, further comprising a detected data analysis unit that specifies intensity of fluorescence emitted from an epidermal layer on the basis of a difference in intensity between fluorescence detected by the light detection unit when the pressure in the housing is the second pressure and fluorescence detected by the light detection unit when the pressure in the housing is the first pressure.

21: The measuring device according to claim 19, further comprising a detected data analysis unit that specifies intensity of fluorescence emitted from the dermic layer on the basis of a difference in intensity between fluorescence detected by the light detection unit when the pressure in the housing is the third pressure and fluorescence detected by the light detection unit when the pressure in the housing is the first pressure.

22: The measuring device according to claim 14, wherein a wavelength of light emitted from the light source is a wavelength within a range capable of detecting advanced glycation endproducts.

23: The measuring device according to claim 14, further comprising another light source that emits near-infrared light or infrared light to the portion of the skin.

24: The measuring device according to claim 14, further comprising a clip for pinching a section of an earlobe,
wherein the skin sampling member is provided in a position capable of pulling the section of the earlobe, which is pinched by the clips, through the suction hole.

25: A measuring method using the measuring device according to claim 14, the method comprising:
removing air from the housing through the exhaust hole to reduce pressure in the housing;
emitting light to a portion of skin that is pulled into the housing through the suction hole in the removing; and
detecting light generated at the portion of the skin being irradiated with the light emitted by the emitting.
